(19) United States
(12) Patent Application Publication
(10) Pub. No.: US 2016/0303226 A1
(43) Pub. Date: Oct. 20, 2016

(54) HEAT-TREATED FORMULATION OF BIFIDOBACTERIUM LACTIS NCC 2818 REDUCES ALLERGIC MANIFESTATIONS

(71) Applicant: NESTEC S.A., Vevey (CH)

(72) Inventors: Annick Mercenier, Bussigny (CH); Guenolee Prioult, Lausanne (CH)

(21) Appl. No.: 15/038,666

(22) PCT Filed: Nov. 4, 2014
(86) PCT No.: PCT/EP2014/073626

§ 371 Date: May 23, 2016

(30) Foreign Application Priority Data
Nov. 25, 2013 (EP) 13194202.1

(51) Int. Cl. A61K 39/35; A23C 9/123; A23C 9/152; A61K 9/00

(52) U.S. Cl. CPC A61K 39/35; A61K 9/0053; A61K 9/0095; A23L 33/135; A23L 33/40; A23C 9/123; A23C 9/152; A61K 2039/522

(57) ABSTRACT

Heat treated Bifidobacterium lactis NCC 2818 reduces the symptoms of allergies in different groups of patients, such as allergies originating from food allergens in young children, or infants, and food, respiratory and cutaneous allergens in children, adults and household pets. The heat treated Bifidobacterium lactis NCC 2818 may be administered alone or in a composition. A reduction of allergy symptoms has been measured, in vivo, in a mouse allergy model.

### FIELD OF THE INVENTION

This invention relates to the use of probiotics, especially Bifidobacterium strains for reducing the symptoms of allergic patients that have been exposed to allergens. Specifically the present invention relates to the use of non-replicating micro-organisms (NRM) to prepare compositions to treat allergic manifestations.

### BACKGROUND TO THE INVENTION

The prevalence of allergic diseases increased rapidly within the last decades. As by now over a third of the worldwide population is afflicted, allergy has been considered as the new epidemic of the industrialized countries. The reasons for the steady increase in allergic diseases are not yet fully understood. Genetic background of the host is a prominent factor, and recently discovered genes have been shown to be associated with respiratory allergies/asthma and skin symptoms [Holloway, J. W. et al. (2010); Genetics of Allergic Disease, J.A.C.I., 125: S81-94]. Environmental factors such as lifestyle, pollution, decreasing family size, and reduction of microbial stimulation of the immune system in early life stage as a consequence of an improved hygienic situation seem to play an important role also.

Allergic sensitization in childhood, especially in early childhood and especially to food allergens, is critical and of highest interest as development of an “allergic phenotype” or “atopy” has been shown to facilitate subsequent sensitization to other allergens. Hence allergies in childhood can be the first step of an allergic cascade leading to multiple allergies later in life, a process commonly referred to as “The Atopic March”. For example, it has been demonstrated in human cohorts that children with persistent food hypersensitivity early in life have a dramatically increased risk to develop allergic rhinitis (hay fever) or asthma later in childhood [Östblom, E. et al. (2008); Phenotypes of food hypersensitivity and development of allergic diseases during the first 8 years of life, Clinical and Experimental Allergy, 38 (8): 1325-1332]. Children with milder forms of food hypersensitivity also have increased risk for development of respiratory allergies but to a lesser degree than children with persistent food hypersensitivity. Therefore, attenuating the severity of food hypersensitivity and management of allergic episodes in children and infancy may be crucial for slowing down the “Atopic March”.

The immune system of infants is actively developing all along the few first years of life. Acting on, preventing, avoiding, managing, reducing or modulating the allergic reactions in such young patients can influence their allergic profile short term but also longer term for later in life. Of course, many adults do have allergies, whether they be food, airborne or contact. The management of allergic symptoms in adults is of utmost importance also.

Prevention of Allergies can be Achieved on Different Levels:

“Primary prevention” is the effect of preventing or reducing the risk of sensitization of patients to allergens, characterized by absence or reduced levels of allergen-specific IgE antibodies. Preventing or reducing sensitization will result in absence or reduction of allergic symptoms upon exposure to the same allergen. By modulating the way a patient becomes sensitized with respect to one allergen or one group of allergens (primary prevention), the subsequent allergic response may also be modulated.

“Secondary prevention” is the effect of modulating the symptoms of allergies, i.e. the occurrence or intensity of the allergic reaction in patient already sensitized to one or several allergens when the patient is re-exposed to said allergen(s). By modulating the occurrence or intensity of the allergic symptoms (secondary prevention), the inconvenience associated with allergies is minimized.

Given these distinct concepts of allergy prevention it may be hypothesized that by virtue of their inherent mechanisms of action, some compounds might act solely at one or at both of these specific levels of prevention. Some may, for example, solely reduce the risk of the sensitization to a specific allergen (primary prevention), while other compounds may solely have an effect on the secondary prevention and reduce the severity of allergic reactions. Other compounds may be able to influence both sensitization and symptoms and thus are effective in promoting primary and secondary prevention.

Food allergens are among the first allergens that infants encounter in their early life: typically, cow's milk proteins may be encountered by infants not receiving exclusive breast-feeding. Milk-proteins are indeed among the most frequently observed causes for food allergy in infancy, followed by eggs, nuts and wheat proteins. In general, food allergies can manifest by cutaneous (rash, eczema, others) and gastrointestinal symptoms (abdominal cramps; pain, especially in the abdomen; vomiting) in infants and young children. Further sensitization and episodes of allergies can also appear when the infant/young child is exposed to a novel food such as cereals, vegetables, fruits, nuts or fish and also to air borne allergens such as pollen, house dust mites and animal dander. Adults are affected to a large extent by contact and respiratory allergies. Recent data from the WHO [Clark, M. J. and. Million, R. P (2009) Allergic rhinitis: market evolution, Nature Reviews, Drug Discovery, 8, p. 271-272.] indicates that up to 30-40% of the world's population suffer from some form of respiratory allergy.

Gastrointestinal Microbiota and the Immune System

Commensal gastrointestinal microbes constitute the earliest and most substantial stimulus for the development of the gut associated lymphoid tissue and associated immune system.

There is solid evidence from epidemiological studies that Western-type living conditions, e.g. reduced consumption of fermented food, substantial use of antibiotics and other drugs, and increased hygiene, are associated with the rise in allergic diseases. The so-called “hygiene hypothesis” thus suggests that a lack of exposure to microbial stimulus early in childhood is a major factor involved in this trend [Von Mutius, E. (2007); Allergies, Infections and the hygiene hypothesis—The epidemiological evidence, Immunobiology, 212(6); 433-9].

Indeed, epidemiological studies have demonstrated an association between the development of allergic diseases and disturbance of the gastrointestinal microbiota. For example, atopic children in Western societies have been reported to be less frequently colonized with lactobacilli or bifidobacteria than healthy children [Suzuki, S., Shimojo, N., Tajiri, Y., Kumemura, M., Kohno, Y. (2007); Differences in the composition of intestinal Bifidobacterium species and the development of allergic diseases in infants in rural Japan, Clin Exp Allergy; 37; 506-511; Ouwehand, A. C., Isolauri, E., He, F., Hashimoto, H., Benno, Y., Salminen, S. (2001); Differences in Bifidobacterium flora composition in allergic and healthy infants, J. Allergy Clin. Immunol. 108; 144-145; Kalliomaki, M., Salminen, S., Arivilommi, H., Kero, P., Koskinen, P., and Isolauri, E. (2001); Probiotics in primary prevention of atopic disease: a randomised placebo-controlled trial, Lancet 357: 1076-9].

The scientific literature reports on the use of probiotics to impact positively on the health of neonates, infants, and children, in particular, with respect to reducing allergy, either as primary prevention or secondary prevention [Prescott, S. L. and Bjorksten B (2007) Probiotics for the prevention or treatment of allergic diseases, J. All. and Clin. Imm. 120, p. 255-262]

Various probiotic cultures or mixes of probiotics have been described for their effect on the allergic immune system: for example, EP1858336 (WO2006697949), describes a mix of probiotics that can decrease the risk of allergies due to wheat flour albumin and globulins (celiac disease) The probiotic mixture was unexpectedly cabable of hydrolyzing gliadin and glutenin fractions which are responsible for celiac disease.

In particular, in a clinical study carried out on a group of 27 infants, aged about 4 and a half months and suffering from atopic eczema, it was reported that after two months of supplementation of an extensively hydrolysed whey infant formula with, either Bifidobacterium lactis CNCM-I-3446 (also known as Bb-12), or Lactobacillus rhamnosus strain GG (ATCC 53103), a significant improvement in the skin condition of the supplemented babies occurred, compared to that of the unsupplemented group. A corresponding decrease in soluble CD4 in serum and eosinophilic protein X in urine in the probiotic supplemented children was also measured [Isolauri, E. T; et al. 2000, Clinical and Experimental Allergy, vol 30, pg 1604-1210].

It has also been demonstrated in a clinical study (with 171 mother infant pairs, double blind placebo controlled) that probiotic supplementation during pregnancy and lactation had a protective effect against sensitization in infants with a high heredity risk due to maternal sensitization [A. Huurre et al. (2008), Impact of maternal atopy and probiotic supplementation during pregnancy on infant sensitization: a double-blind placebo-controlled study, Clin. Exp. Allergy; 38; 1342-1348].

There still remains a need for specifically reducing allergic reactions and symptoms in allergic infants, children and adults. This is also true for animals, in particular pets who may suffer from food, airborne or contact allergies. This is especially important when considering the maturation of both the intestinal and immune systems occurring in young children or mammals and when considering the multiplicity of novel allergens that the young children are exposed to, especially around weaning.

There is a need to bring relief in the symptoms of food, contact and airborne allergies in mammals, in particular in infants, young children and adults as well as companion animals that have a history of allergic episodes and/or are allergic.

There is a need to provide a complete nutritional composition that not only brings a variety of nutrients but also reduces the severity of allergic reactions.

There is a need to provide a nutritional composition that modulates the allergic reaction of young patients suffering from light to moderate forms of allergies, as these patients have special needs dictated by the incomplete maturity of their intestinal and immune systems.

These patients may also be unable to tolerate stringent pharmaceutical molecules, and need, for example, nutritional intervention to modulate the immune system.

There is a need to provide a nutritional composition that can modulate the allergic reaction of young patients around weaning when the intestinal tract undergoes substantial modification and when new solid foods, potentially containing new allergenic proteins, are introduced and hence the patient is particularly susceptible to the sensitization to food allergens.

There is a need to provide a nutritional composition to manage or reduce symptoms of respiratory allergy (i.e. air-borne allergies) in children and adults upon exposure to seasonal (pollen) or perennial (house dust mite, cockroach, pet dander, molds & fungi) airborne allergens.

In their search for effective treatments for allergic symptom management, the present inventors have investigated a number of probiotics, and in particular Bifidobacterium lactis NCC 2818.

The prior art generally teaches that heat treatment of probiotics leads to a partial or complete loss of their health beneficial properties. [Verdu et al., (2004), Gastroenterology, 127, p. 826 ff., Rousseaux, 2007, Nature Medicine, 13, p. 35ff; and Kamiya et al., (2006), Gut, 55,191]. Only in exceptional cases, some health benefits tested were maintained.

Non-replicating probiotic micro-organisms have the advantage that they are far easier to handle than their live counterparts. Additionally, they are far more storage stable and need less stringent packaging conditions.

### SUMMARY OF THE INVENTION

The present invention relates to heat treated non replicating B. lactis NCC 2818 for reducing the symptoms in patients having allergies triggered by food or air borne or contact allergens.

The patients may be human subjects, in particular adults, young children or infants. The patients may be animals. The symptoms may be gastro-intestinal, cutaneous, ocular or respiratory or a combination thereof.

The invention is especially effective in already sensitized subjects.

The heat treated non replicating B. lactis NCC 2818 may be administered as a nutritional or topical composition. The nutritional composition may be a food supplement, a milk fortifier, or any milk support used during trophic feeding, an infant formula, a growing-up milk (powder or liquid), Non-replicating B. lactis NCC 2818 according to claim 3, wherein said nutritional composition is a food supplement, a human milk fortifier, or any milk support used during trophic feeding, an infant formula, a growing-up milk (powder or liquid), a nutritional dairy-based beverage (powder or liquid), a milk-based drink, a spoonable dairy-based food, a spoonable yoghurt-based food, a cereal-based milk drink, baby cereals, a yoghurt, a baby meal, a spoonable cheese-based food, dairy and fruit drink, a smoothy, a pudding, a snack or a biscuit or other bakery items. The composition may be an especially adapted hypo-allergenic nutritional composition, including an HA infant formula (partially or extensively hydrolyzed formulas). This formula may be any kind of cow's milk derived formulae, soy based formulae or free amino acid formulae.

The heat treated non-replicating B. lactis NCC 2818 may be administered in a daily dose of the equivalent of between 10⁵ and 10¹⁰ colony forming units (cfu) per day.

Administration of heat treated non-replicating B. lactis NCC 2818 restores the immune balance in allergic individuals and thus can be effective for different types of allergy.

### BRIEF DESCRIPTION OF THE DRAWINGS

FIG. 1: Schematic description of OVA food allergy mouse model. 6-week-old BALB/c mice (10-15 animals/group) were sensitized with 20 mg OVA and 10 μg/mouse of Cholera toxin as an adjuvant for 7 weeks (Sensitization). Then an oral challenge with 100 mg of OVA was performed (Challenge). Live B. lactis NCC 2818 (Experiment 1) or non replicating heat treated B. lactis NCC 2818 (heat treated at 120° C., 15 seconds) (Experiment 2) was given to sensitized mice via drinking water (5×10⁸ equivalent CFU/ml) either during the sensitization-challenge period day 1-50 to study the effect of the probiotic on primary prevention, or starting at the end of the sensitization phase (day 43-49) to study the effect of the probiotic on symptom management.

FIG. 2: Effect of live and heat treated B. lactis NCC 2818 on symptoms of food allergy as measured in the ovalbumin (OVA) food allergy mouse model of Example 2. Symptoms are measured in mice receiving live (Experiment 1) or non-replicating heat-treated (Experiment 2) B. lactis NCC 2818 via drinking water after sensitization in the management phase, during the last week before challenge. The symbols , ▪, ▴ and ▾ represent individual mice in the separate groups. Groups that are significantly different (p<0.05) are indicated by *. The negative control group () was not sensitized with OVA, but received only cholera toxin. The positive control group (▪) was sensitized, but not treated with B. lactis NCC 2818. In Experiment 1, the group labelled “Prevention” (▾) was treated with live B. lactis NCC 2818, during the entire experiment, and the group labelled “Management” (▴) was treated with live B lactis NCC 2818 only during the last week of the experiments. In Experiment 2, the group labelled “Prevention” (▾) was treated with heat treated B. lactis NCC 2818 during the entire experiment, and the group labelled “Management” (▴) was treated with heat treated B. lactis NCC 2818 during only the last week of the experiments. Thus, experiments 1 and 2 are similar, with live and heat treated B. lactis respectively.

FIG. 3: Levels of Murine Mast-Cell Protease 1 (MMCP-1) in mouse sera 4 hours after challenge in the OVA food allergy mouse model. Experiments 1 and 2 (see FIG. 2). In Experiment 1, the group labelled “Prevention” (▾) was treated with live B. lactis NCC 2818, during the entire experiment, and the group labelled “Management” (▴) was treated with live B. lactis NCC 2818 only during the last week of the experiment. In Experiment 2, the group labelled “Prevention” (▾) was treated with heat treated B. lactis NCC 2818 during the entire experiment, and the group labelled “Management” (▴) was treated with heat treated B. lactis NCC 2818 during only the last week of the experiment. The symbols , ▪, ▴ and ▾ represent individual mice in the separate groups. Groups that are significantly different (p<0.05) are indicated by *.

### DETAILED DESCRIPTION OF THE INVENTION

### Definitions

In this specification, the following terms have the following meanings:

“Patients” mean mammals, including humans and animals. Specifically, the term “patients” means animals, infants, children, teenagers or adults suffering from allergic manifestations, or who are at risk of developing allergic manifestations and/or who are sensitized to allergens. Animals include especially domestic animals, such as cats and dogs.

Allergic children are those children, babies or infants having experienced at least one episode of allergic reaction—light, moderate or severe—to a food or airborne allergen.

For the purpose of this document a “complete nutritional composition” is a composition that comprises a significant amount, usually 20% or more, of the major nutritional nutrients recommended for a given age. Such major nutrients are usually provided in quantity and proportion such as to fulfil 20% or more of the specific recommended nutrient's doses for a given age, when used in adequate quantity for providing the recommended caloric intake for a given age. A complete nutritional composition usually comprises a source of protein, a source of lipid, a source of carbohydrates in a balanced proportion that meets the general recommendation for a given age. It usually also includes micronutrients such as vitamins and minerals, as well as a source of essential amino acids and a source of essential fatty acids. It is however understood that a complete nutritional composition may not comprise all specific nutrients, or all recommended amounts, to fulfil all nutritional needs of an infant or young child. A complete nutritional composition excludes compositions comprising merely Bifidobacterium lactis NCC 2818, or Bifidobacterium lactis NCC 2818 in a predominant proportion.

“Symptoms of allergies” generally include symptoms triggered by allergens. Such symptoms include cutaneous (redness of skin, rash, itchiness, dermatitis, eczema), ocular (itching and watering of the eyes), gastrointestinal (constipation, abdominal pain, cramps, vomiting diarrhea), respiratory (itching of the nose, nasal congestion, rhinitis, asthma) and, in severe cases, systemic (dizziness, mental confusion, anaphylaxis) manifestations.

“Primary prevention of allergies” means all measures aiming at avoidance or reduction of allergic (immunological) sensitization for example prevention of induction, or reduction of specific IgE antibodies.

“Secondary prevention of allergies” means prevention or reduction of the development of allergic disease/allergic symptoms in a sensitized individual.

“Weaning period” is the period during which infants are adapting from pure liquid nutrition to solid or semi-solid food, and adapting from quasi unique food type (generally mother milk or infant formula) to a variety of foods.

“Sensitization” means induction/development of allergen-specific IgE antibodies, with or without the manifestation of symptoms.

“Probiotic” means microbial cell preparations or components of microbial cells with a beneficial effect on the health or well-being of the host [Salminen S, Ouwehand A. Benno Y. et al (1999) “Probiotics: how should they be defined” Trends Food Sci. Technol.:10 107-10]. The definition of probiotic is generally admitted and in line with the WHO definition. The probiotic can comprise a unique strain of micro-organism, of a mix of various strains and/or a mix of various bacterial species and genera. In case of mixtures, the singular term “probiotic” can still be used to designate the probiotic mixture or preparation. The probiotic may or may not include the culture supernatant. For the purpose of the present invention, micro-organisms of the species Bifidobacterium lactis are considered as probiotics.

“Prebiotic” generally means a non digestible food ingredient that beneficially affects the host by selectively stimulating the growth and/or activity of micro-organisms present in the gut of the host, and thus attempt to improve host health.

“Airborne allergies” and “airborne allergens” are used as synonyms of respiratory allergies and respiratory allergens.

Cutaneous allergies or skin allergies are allergies whose symptoms include redness, rash or irritation of the skin.

Bifidobacterium lactis (B. lactis) strain NCC 2818 (Nestle Culture collection) is the B. lactis strain with the international identification reference CNCM-I-3446 (Collection Nationale de Cultures de Microorganismes at Institute Pasteur, Paris, France). CNCM identifications refer to the Collection Nationale de Cultures de Microorganismes at Institut Pasteur, 22 rue du docteur Roux, 75724 Paris, France. This strain is also known in the art as BB-12© and commercially available from Chr. Hansen, Horsholm, Denmark In the current invention, Bifidobacterium lactis strain NCC 2818 is known as B. lactis.

“Non-replicating” micro-organisms include micro-organisms, e.g., probiotic bacteria and dairy starter cultures, which have been heat treated or killed by any other process, such as, for example, radiation or extrusion, that leads to non-replicating micro-organisms that have a similar capacity to modulate immune reactions in patients, as the micro-organisms treated by the heat process. This includes micro-organisms that are inactivated, dead, non-viable and/or present as fragments such as DNA, metabolites, cytoplasmic compounds, and/or cell wall materials.

“Non-replicating” means that no viable cells and/or colony forming units can be detected by classical plating methods. Such classical plating methods are summarized in the microbiology book: James Monroe Jay, Martin J. Loessner, David A. Golden. 2005. Modern food microbiology. 7th edition, Springer Science, New York, N.Y. 790 p. Typically, the absence of viable cells can be shown as follows: no visible colony on agar plates or no increasing turbidity in liquid growth medium after inoculation with different concentrations of bacterial preparations (non replicating′ samples) and incubation under appropriate conditions (aerobic and/or anaerobic atmosphere for at least 24 h).

The invention concerns the administration of a non-replicating probiotic, in particular, heat-treated B. lactis NCC 2818 to mammals having allergies triggered by food, airborne or contact allergens for reducing their symptoms. The inventors have evidenced that consumption of heat-treated B. lactis NCC 2818 preparations leads to reduced symptoms of food allergy in a group of young adult mice receiving a nutritional composition containing said preparation of B. lactis. This is demonstrated when an allergic reaction (challenge) is induced after sensitization. The model mimics food allergy in humans, when humans are naturally sensitized to food allergens and further re-exposed to said allergens. The bacterial strain B. lactis NCC 2818 hence shows a protective effect when heat treated under specific conditions.

Surprisingly this effect was not accompanied by a reduction of the sensitization of the mammals to allergens (i.e. no primary prevention of allergies was evidenced).

Details of the mode of administration of the probiotic, and the effect thereof are given in the following paragraphs.

Heat Treatment of the B. lactis NCC 2818 to Render it Non-Replicating

Methods described in the art, for example in European Patent publication EP 2251020 may be used to render the B. lactis non-replicating. The temperature ranges that may be used in the current invention are from 90° C. to 150 C.°, and the duration of treatment may be from 5 seconds to 60 minutes. Typically, the longer durations or time are used for lower temperatures and the shorter time for higher temperatures. For example, the heat-treatment may be carried out in the range 145° C.-150° C., for 3-10 seconds, or 90° C.-100° C., for 50 minutes.

Heat treated bacteria have the advantage that they may be present or added into products in which health promoting bacteria (probiotics) are difficult to maintain alive (for example, in liquid products, products with a long shelf life, products with high water activity and non refrigerated products).

Heat treated bacteria may also be added into products dedicated to immunocompromised patients for whom the risk of bacteremia may be high after consumption of live bacteria. They may also be included in ocular products (into which live strains may not be introduced).

Doses of Probiotic

Obviously, non-replicating micro-organisms do not form colonies. Consequently, 1×10⁴ to 1×10¹² cfu in the context of the current invention is to be understood as the amount of non replicating micro-organisms that is obtained from 10⁴ and 10¹² cfu replicating bacteria. This includes micro-organisms that are inactivated, non-viable or dead or present as fragments such as DNA or cell wall or cytoplasmic compounds. In other words, the quantity of micro-organisms which the composition contains is expressed in terms of the colony forming ability (cfu) of that quantity of micro-organisms as if all the micro-organisms were alive irrespective of whether they are, in fact, non replicating, such as inactivated or dead, fragmented or a mixture of any or all of these states.

The heat treated non-replicating B. lactis NCC 2818 may be administered in a daily dose of the equivalent of between 10⁵ and 10¹⁰ colony forming units (cfu) per day. The lower doses are intended for new-born babies and the higher doses for adults.

The daily dose can be administered in one administration per day or alternatively in 2, 3 or 4 administration per day. The daily administration (or 2 times daily or 3 or 4 times daily) may be repeated over a long period of time (2, 3, 5, 7, 10, 15, 21, 30 days or more). Such repeated modes of administration are believed to deliver stronger and more sustainable effect.

B. lactis NCC 2818 may be present in a composition administered to the mammal in a wide range of percentages provided that it delivers the positive effect described.

Thus the amount of probiotic present per gram of dry composition for administration may vary as long as the daily doses described above are respected. For example, the B. lactis NCC 2818 may be present in an infant formula composition in an amount equivalent to between 1×10² and 1×10¹¹ cfu/g of dry composition, preferably 1×10⁴ to 1×10⁹ cfu/g of dry composition.

Method of Administration

The heat treated non replicating B. lactis NCC 2818 of the invention can be administered orally or topically to the mammal. If administration is oral, it may be administered pure or resuspended in water or mother's milk for young mammals for example, or administered as a food supplement or as an ingredient in an infant milk formula, or other nutritional composition. An infant formula may be an infant “starter formula” if probiotic administration starts before the infant is 6 months old, or a “follow-on formula” if the infant is older than 6 months.

The formula may also be a hypoallergenic (HA) formula in which the cow milk proteins are (partially or extensively) hydrolysed. The formula may also be based on soy milk or a non-allergenic formula, for example one based on free amino acids An example of such HA formula is given in Example 3.

If the administration is topical, the heat treated non replicating B. lactis NCC 2818 may be administered in the form of a cream, gel, gel-cream, serum, lotion, milk, or aqueous solution, or ointment. Alternatively, the heat-treated B. lactis NCC 2818 may be administered topically by impregnating a compress with a suspension of the non-replicating probiotic and applying this to the area of skin or mucosa to be treated.

If the young mammal is above 6 months of age, the heat-treated probiotic may be administered in a growing-up milk (powder or liquid), a nutritional dairy-based beverage (powder or liquid), a milk-based drink, a spoonable dairy-based food, a spoonable yoghurt-based food, a cereal-based milk drink, baby cereals, a yoghurt, a baby meal, a spoonable cheese-based food, dairy and fruit drink, a smoothy, a pudding, a snack or a biscuit or other bakery items.

An example of a powdered milk formulation for children is given in Example 4. Example 5 is an example of a yoghurt composition suitable for children or adults. The composition may be in the form of a nutritional or pharmaceutical composition or food supplement for adults suffering from allergies.

If administration is to an animal, it may be in the form of an animal feed (dry or wet food) or a topical formulation for animals (for example, a spray or lotion for treating allergy-associated skin irruptions or lesions).

As the B. lactis NCC 2818 of the invention is non-replicating, it may be conveniently included in products which are shelf stable, freeze or spray dried products, or products which may have been produced by extrusion, agglomeration, an aseptic process or retort.

Administration Period

The administration of heat treated B. lactis NCC 2818 can begin as soon as there is suspicion or evidence that the patient is sensitized. This may be, for example, that an allergic condition has been confirmed in an allergy test, or that early symptoms/signs of allergy have occurred in infants, children or adults. It can be more specifically targeted to and administered during the weaning period and/or up to 12 months thereafter. The weaning period is indeed important in regard to the invention as the infants are exposed to a variety of foods during the weaning period, while still undergoing maturation and modification of their immune system and their gastro-intestinal tract. Effective control of the allergic response is therefore of particular importance during that period. Patients, who become sensitized to new allergens as adults (for example, after changing country) may also benefit from control of allergic responses by administration of heat-treated B. lactis NCC 2818.

In the population that is sensitized to airborne allergens, whether this population be infants, children, teenagers or adults, the heat treated B. lactis NCC 2818 may be administered before the pollen season begins or before and during the pollen season, or throughout the year in case of perennial allergies such as those caused by dust mite or pet dander.

In the patient population (infants, children teenagers or adults) that suffers respiratory, or contact, or food allergies, the heat treated B. lactis NCC 2818 may be administered upon appearance of the first symptoms.

The duration of the administration may vary. While positive effects are expected with relatively short duration of administration (for example, daily administration during one to two months for young allergic infants), longer durations are believed to provide an enhanced effect, or, at least, to maintain the effect in older infants (for example, a duration of three, five, eight or 12 months) or in young children (for example, a duration up to, for example the age of 4 years.

Administration in adults may have a duration of weeks, months or even years. The factors influencing the duration of administration are the severity of the allergic symptoms, the effectiveness of the treatment on the particular subject, the convenience of administration and the wishes or needs of the patient.

For administration to animals, the corresponding durations apply.

The period of administration can be continuous, or discontinuous. Continuous administration is preferred for a more sustained effect. However, it is speculated that a discontinuous pattern (for example, daily administration during one week per month, or during alternate weeks) can induce positive effects. For young infants, the administration may coincide with the number of daily feeds, especially if the B. lactis NCC 2818 is incorporated into the infant formula, i.e. about four to about six times daily for infants less than one year old, the number gradually reducing with age. For infants older than one year, the administration may be less frequent, for example once or twice a day. For toddlers and young children, the administration may be daily or weekly (to be taken minimum twice a week). For adults, administration may also be daily or on alternate days. The period of administration should lead to the administration of an effective daily dose of non-replicating microorganisms. An effective daily dose is defined as the equivalent of between 10⁵ and 10¹⁰ colony forming units (cfu) per day.

For animals, the same administration frequencies may generally be used.

The administration to the allergic infant, child or adult may be continued as long as symptoms last, and before or during the pollen season in case of seasonal allergies.

In the case of perennial respiratory allergies (house dust mite, pet dander) as long as the symptoms persist.

Administration with Other Compounds

The heat-treated B. lactis NCC 2818 may be administered with one or more additional probiotics. Preferably, the probiotic may be selected for this purpose from the group consisting of Bifidobacterium, Lactobacillus, Lactococcus, Enterococcus, Streptococcus, Kluyveromyces, Saccharoymces, Candida, in particular selected from the group consisting of Bifidobacterium longum, Bifidobacterium lactis, Bifidobacterium animalis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1.3724), Enterococcus faecium SF 68 (NCC2768; NCIMB10415), and mixtures thereof.

It is speculated that the administration of the combination of the heat treated and live strain may provide a complementary (if not synergistic) effect in the prevention and management of allergy in infants, young children and adults.

The B. lactis NCC 2818 can be administered alone (pure or resuspended in water or milk, including breast milk for example) or in a mixture with other compounds (such as dietary supplements, nutritional supplements, medicines, carriers, flavours, digestible or non-digestible ingredients). Vitamins, minerals and supplements for pregnant women are examples of typical dietary supplements. In a preferred embodiment, the composition is administered together with other compounds that enhance the described effect on the immunity of the progeny. Such synergistic compounds may be carriers or a matrix that facilitates the B. lactis NCC 2818 delivery to the intestinal tract of the (young) mammal. Such compounds can be other active compounds that synergistically, or separately, influence the immune response of the subject and/or potentiate the effect of the probiotic or non-replicating microorganism, such as prebiotics.

The prebiotics that may be used in accordance with the present invention are not particularly limited and include all food substances that promote the growth of probiotics or health beneficial micro-organisms in the intestines. Preferably, they may be selected from the group consisting of oligosaccharides, optionally containing fructose, galactose, mannose; dietary fibers, in particular soluble fibers, soy fibers; inulin; or mixtures thereof. Preferred prebiotics are fructo-oligosaccharides (FOS), galacto-oligosaccharides (GOS), isomalto-oligosaccharides (IMO), xylo-oligosaccharides (XOS), arabino-xylo oligosaccharides (AXOS), mannan-oligosaccharides (MOS), oligosaccharides of soy, glycosylsucrose (GS), lactosucrose (LS), lactulose (LA), palatinose-oligosaccharides (PAO), malto-oligosaccharides, gums and/or hydrolysates thereof, pectins and/or hydrolysates thereof.

The daily doses of carbohydrates, and all other compounds administered with the B. lactis NCC 2818 should always comply with the published safety guidelines and regulatory requirements. This is particularly important with respect to the administration to young infants, under one year old.

In one embodiment, the heat treated B. lactis NCC 2818 is administered in a composition further comprising an apple extract comprising polyphenols. The apple extract can help reducing the symptoms of allergies originating from food, airborne or contact allergens in patients having allergies triggered by said allergens. As such, the apple extract acts in a synergistic way with the heat treated B. lactis NCC 2818 in order to modulate, reduce, or attenuate allergies in patients having food allergies. In one embodiment, such composition is used for baby food and/or baby cereals that naturally represent a suitable carrier for the composition. In a further embodiment, the baby food or baby cereals comprises apple extracts or material from apple

In one embodiment, the heat treated B. lactis NCC 2818 is administered in the form of a nutritional composition which, preferably, comprises a source of protein. Dietary protein is preferred as a source of protein. The dietary protein may be any suitable dietary protein, for example animal proteins (such as milk proteins or meat proteins), vegetable proteins (such as soy proteins, wheat proteins, rice proteins or pea proteins), a mixture of free amino acids, or a combination thereof. Milk proteins such as casein and whey proteins are particularly preferred.

The composition may also comprise a source of carbohydrates and/or a source of fat.

The lipid making up the fat source may be any suitable fat or fat mixture. Vegetable fat is particularly suitable, for example soy oil, palm oil, coconut oil, safflower oil, sunflower oil, corn oil, canola oil, lecithin and the like. Animal fat such as milk fat may also be added if desired.

An additional source of suitable carbohydrate may be added to the nutritional composition. Any suitable carbohydrate may be used, for example sucrose, lactose, glucose, fructose, corn syrup solids, maltodextrin, or a mixture thereof. Additional suitable dietary fibre may also be added. The dietary fibre may be from any suitable origin, including for example soy, pea, oat, pectin, guar gum, acacia gum, fructooligosaccharide or a mixture thereof. Suitable vitamins and minerals may be included in the nutritional composition in an amount to meet the appropriate guidelines for the targeted population (infants, children, adults).

One or more essential long chain fatty acids (LC-PUFAs) may be included in the composition. Examples of LC-PUFAs that may be added are docosahexaenoic acid (DHA) and arachidonic acid (AA).

Effect of the Probiotic Administration

The consumption of B. lactis NCC 2818 leads to reduced symptoms of food allergy. In an experimental animal model of food allergy (Example 1), the inventors demonstrate that B. lactis NCC 2818 heat treated by high temperatures for a short time (for details on suitable temperatures and times see published patent application EP 2251020) reduced allergy symptoms. In the mouse model experiments, both allergy symptoms and expression levels of inflammation biomarkers were measured in mice in which an allergic reaction (challenge) is induced after sensitization. The model mimics food allergy in humans, when humans are naturally sensitized to food allergens and further re-exposed to said allergens. The heat treated bacterial strain B. lactis NCC 2818 hence shows a protective effect.

Allergy Symptoms are Reduced Upon Administration of Heat Treated B. lactis NCC 2818

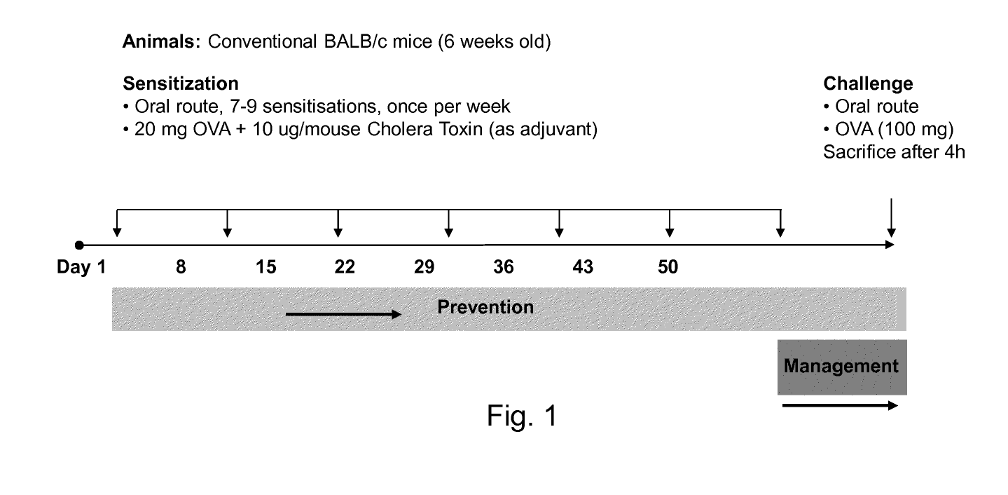

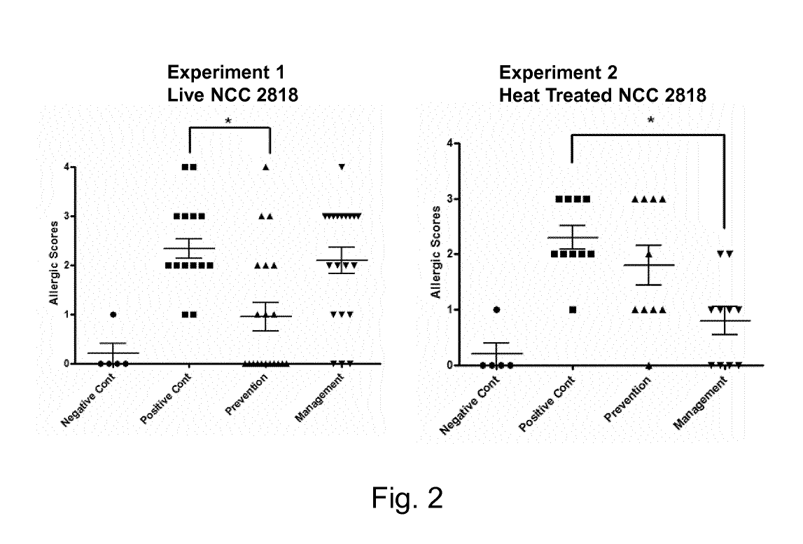

Administration of heat treated B. lactis NCC 2818 leads to the reduction of allergic symptoms. This is illustrated in a set of experiments using an animal model of food allergy in Example 1. The model design is illustrated in FIG. 1 and the experimental results are illustrated in FIG. 2.

In this model, BALB/c mice are sensitized at weekly intervals with orally administered ovalbumin (OVA—allergen)+cholera toxin (CT—adjuvant) during 7 continuous weeks (see FIG. 1). Oral challenge with a large dose of OVA one week after the last sensitization leads to allergic clinical symptoms such as diarrhoea, scratching episodes, bristled fur, cyanosis and loss of mobility.

The effect of both live B. lactis NCC 2818 (Experiment 1) and non replicating heat-treated B. lactis NCC 2818 (Experiment 2) on allergic manifestations induced by oral re-exposure to a high dose of the allergen in sensitized animals, was measured. The impact of the probiotic administration on the development of sensitization to the allergen was also evaluated. The results are shown in FIG. 2. It is striking that the group receiving live B. lactis NCC 2818 (Experiment 1) during the management phase (▴) had no reduction in symptoms, compared to the positive control group (▪), whereas the group receiving the heat treated B. lactis NCC 2818 (Experiment 2), group (▴) during the same period had a considerably lower allergic score (i.e. less severe symptoms) than the positive control group (see Example 1 for details of allergic score calculation).

Interestingly, the live strain impacts more in the prevention phase (sensitization to allergen) compared to the heat treated strain. The animals receiving live B. lactis NCC 2818 in the prevention phase (Experiment 1, group ▾) have a significantly lower allergic score than the positive control group (▪), whereas the group receiving the heat treated B. lactis NCC 2818 in the prevention phase (Experiment 2, group (▾)) were not significantly different from the positive control group.

This seems to indicate that heat treated B. lactis NCC 2818 has little or no effect on the sensitization of the animals to the allergens. Indeed, primary prevention seems not to be achieved by administration of heat treated B. lactis NCC 2818. Thus, the subjects are still sensitized to the food allergens, but develop less symptoms of allergy when re-exposed to the sensitizing allergen. Without being bound to the theory, it is believed that the effect therefore does not reduce the natural immune defences (which have a positive effect for the patient) but simply reduces the expression of the symptoms of allergy.

We note that in trial reported in Isolauri, E. T; et al. 2000, mentioned above, supplementation of infant formula with the live strain B. lactis NCC 2818 was shown to reduce allergic symptoms in babies when measured after two months of feeding. The inventors note that, in the experiments of Example 1, the reduction in allergy symptoms was measured after only one week of treatment of symptoms in young adult mice. At this time point, no beneficial effect on allergic symptom reduction was seen for the live strain. However, it is entirely possible that such a beneficial effect may be seen after a longer time period. The experimental set up of Example 1 demonstrates the strong effect of heat treated B. lactis NCC 2818 (which is better than live B. lactis NCC 2818) without excluding the possibility that live B. lactis NCC 2818 could be effective in a different experimental setting.

The current set of experiments are indeed a novel observation where a live probiotic strain that impacts the sensitization process in vivo shows improved efficacy in the management of allergic symptoms. upon heat treatment, and, as a consequence,

Administration of Heat Treated B. lactis NCC 2818 Decreases Levels of Serum MMCP-1:

Administration of heat treated B. lactis NCC 2818 impacts beneficially on allergic manifestations, as indicated by a reduction in the serum levels of an immunological marker mouse mast-cell protease 1 (MMCP-1) that is released by mast cells, during mast cell degranulation, at allergen challenge. MMCP-1 is a chemical mediator, like histamine, that causes inflammation and tissue damage.

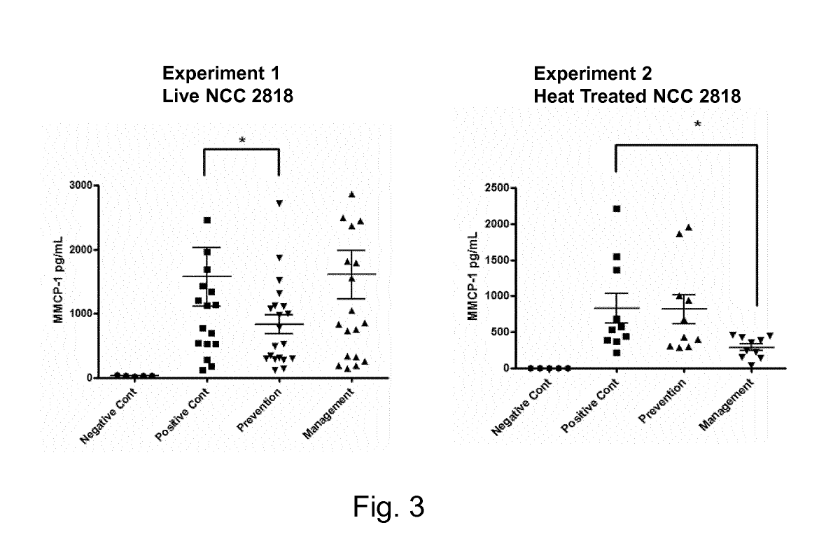

In Example 1, OVA challenge led to a strong increase of serum levels of MMCP-1 in the positive (▪), compared to the negative () control group (see FIG. 3). No significant modulation of MMCP-1 serum levels was observed in mice treated with live B. lactis NCC 2818 (▴) compared to the positive control group (Experiment 1) in the management phase of allergic symptoms. However, in mice treated with the non-replicating heat treated B. lactis NCC 2818 (Experiment 2, group (▴)), there was a significant reduction in levels of MMCP-1 after challenge (p=0.02).

The live B. lactis NCC 2818 strain was effective in inhibiting MMCP-1 levels when administered during the prevention phase (Experiment 1, group ▾), whereas the heat treated B. lactis NCC 2818 was not (Experiment 2, group ▾).

These results are consistent with the allergic score data. The heat treated B. lactis NCC 2818 has no effect on the sensitization of the animals to the allergens and, therefore, does not reduce the natural immune defences. The animals receiving the heat treated B. lactis NCC 2818 strain develop less allergy symptoms upon allergen re-exposure.

The allergic score and immunological data suggest that administration of heat treated B. lactis NCC 2818 restores the immune balance in allergic individuals. Thus administration of heat treated B. lactis NCC 2818 to patients suffering from allergic manifestations reduces the severity of symptoms. The symptoms may be diarrhea, skin irritation, ocular or respiratory symptoms, or combinations thereof. The symptoms themselves may be accompanied by release of biochemical mediators, such a tryptase, chymase, histamine, cytokines and leukotrienes.

Thus, administration of heat treated B. lactis NCC 2818 is effective for the treatment of different allergic disorders triggered by a wide variety of allergens (food, airborne, cutaneous).

Administration of heat treated B. lactis NCC 2818 may also inhibit immune responses to allergic triggers in different mammalian species. Thus, the administration may be to reduce symptoms in species such as cats and dogs that are known to develop allergic reactions.

Target Group

The invention is suitably targeted for different categories of patients: these include either, relatively young patients that develop symptoms to food allergens, or young patients and adult patients that have allergic manifestations to food, airborne or contact allergens. Other mammalian species (especially household pets, such as cats or dogs) that react to similar allergens could also benefit from administration of heat treated B. lactis NCC 2818.

In one embodiment of the invention, for food allergies, the patients are sufficiently young (below 2-3 years old) so that their immune system and/or their gastro-intestinal tract are still maturing. In such patients, the effect of the administration of heat treated B. lactis NCC 2818 can be more intense or more rapid than in older patients.

According to certain embodiments of the invention, the target group may be allergic children, or infants. In one embodiment of the invention, the children or infants have declared severe allergies to food allergens and/or have experienced more than one moderate or severe episode of food allergy, and have displayed allergic symptoms.

The food allergens encompassed by the present invention can include all types of allergens naturally occurring or usually occurring in food, especially food for young humans (e.g. infants and children).

The target group may be human adults suffering from allergy.

The target group may be children and adults that suffer from respiratory allergies. The respiratory allergens encompassed by the present invention can include all types of allergens that are airborne, can be seasonal (pollen) or perennial (house dust mite, cockroach, pet dander, molds and fungi) and contact allergens.

The target group may be other mammalian species such as household pets (cats, dogs) that may develop different manifestations of allergic symptoms.

### Example 1

The experimental set up is shown in FIG. 1. 6-week-old conventional female BALB/c mice (10-15 animals/group) (Harlan Laboratories, Lyon, France) were sensitized via the oral route by a gavage at weekly intervals with 20 mg of ovalbumin (OVA) (Fluke; Sigma, Buchs, Switzerland) and 10 μg/mouse of Cholera toxin (CT) as an adjuvant (List Biological Laboratories, Campbell, Calif., USA) for 7 weeks. One week after the last sensitization, an oral challenge with 100 mg of OVA was performed (on day 50). Mice were fed with B. lactis NCC 2818 (live or heat treated) in their drinking water, at a dose of 5×10⁸ colony forming units per ml (cfu/ml) in drinking water. For the heat treated B. lactis NCC 2818, obviously, no viable colonies can be formed and, thus, the dose 5×10⁸ cfu/ml was the amount equivalent to 5×10⁸ cfu/ml live strain. The nutritional intervention was either during the sensitization period (day 1-50) to test the effect of the probiotic for primary prevention, or, starting at the end of the sensitization phase (day 43-49) to test the efficacy of the strain for symptom management/treatment.

Starting thirty minutes after challenge, mice were individually observed during 30 min. Clinical symptoms were recorded and quantified as follows (Allergic Score): 0: no symptoms, less than 4 episodes of scratching; 1: 4-10 episodes of scratching around the nose and head, no diarrhoea; 2: more than 10 episodes of scratching or bristled fur and immobility or soft stool; 3: diarrhoea or laboured respiration or cyanosis; 4: diarrhoea in combination with immobility after prodding, bristled fur, laboured respiration or cyanosis; 5: anaphylaxis.

The results are shown in FIG. 2. Experiment 1 shows the mice treated with live B. lactis NCC 2818 and Experiment 2 shows the mice treated with heat treated B. lactis NCC 2818.

Four hours after challenge mice were sacrificed (cervical dislocation), and blood was taken and frozen in liquid nitrogen.

Serum Murine Mast Cells Protease 1 MMCP-1

Murine mast cells protease 1 (MMCP-1) was quantified in mouse serum by ELISA, purchased from Moredun Scientific (Penicuik, Scotland) according to the manufacturer's instructions. The MMCP-1 concentration was obtained by converting OD values into pg/mL using a polynomial standard curve. Results are shown in FIG. 3 Each symbol represents one mouse as stated in the legend and statistically different groups are indicated by * (p<0.05).

### Example 2

An example of the composition of a hypoallergenic starter infant formula for use according to an embodiment of the present invention is given below. This composition is given by way of illustration only. The protein source is a conventional mix of whey protein and casein.

| Nutrient | per 100 kcal | per litre |
|---|---|---|
| Energy (kcal) | 100 | 670 |
| Partially hydrolysed Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Prebiotic (100% GOS) (g) | 0.64 | 4.3 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |

| | |
|---|---|
| Heat treated B. lactis NCC 2818 | Equivalent 2 × 10⁷ cfu/g of powder |

### Example 3

Example of a milk powder intended for children comprising heat treated B. lactis NCC 2818. This composition is an illustration of the invention. The protein source is a conventional mix of whey protein and casein

| Nutrient | per 100 kcal | per litre |
|---|---|---|
| Energy (kcal) | 100 | 670 |
| Protein (g) | 3.66 | 24.5 |
| Fat (g) | 4.52 | 30.2 |
| Linoleic acid (g) | 0.69 | 4.6 |
| α-Linolenic acid (mg) | 73 | 490 |
| Carbohydrates (g) | 11.18 | 74.9 |
| Prebiotic (100% GOS) (g) | 0.64 | 4.3 |
| Na (mg) | 49 | 330 |
| Ca (mg) | 183 | 1220 |
| Vitamin A (μg RE) | 77 | 520 |
| Vitamin D (μg) | 0.99 | 6.6 |
| Vitamin K1 (μg) | 4.7 | 32 |
| Vitamin C (mg) | 9 | 60 |
| Niacin (mg) | 1.6 | 11 |
| Folic acid (μg) | 39 | 260 |
| Fe (mg) | 1.5 | 10 |
| Zn (mg) | 1.2 | 7.9 |

| | |
|---|---|
| Heat treated B. lactis NCC 2818 | Equivalent 2 × 10⁸ CFU/g of powder |

### Example 4

An example of the composition of a yoghurt drink given to children and adults according to an embodiment of the present invention. This composition is an illustration only. The protein source is a conventional mix of whey protein and casein

| Nutrient | per 120 kcal |
|---|---|
| Energy (kcal) | 120 |
| Protein (g) | 7.8 |
| Fat (g) | 3.4 |
| Carbohydrates (g) | 14.4 |
| Ca (mg) | 502 |
| Vitamin C (mg) | 17 |
| Vitamin D (IU) | 85 |
| Vitamin K1 (μg) | 4.7 |
| Zinc (mg) | 2 |
| Magnesium (mg) | 29 |
| Heat treated B. lactis NCC 2818 | Equivalent of 7 × 10⁸ |
| | cfu/g of yoghurt |

1. A method for reducing the symptoms of an allergy in a patient comprising the step of administering to the patient having an allergy a composition comprising non-replicating Bifidobacterium lactis NCC 2818 (B. lactis NCC 2818, B. lactis CNCM-I-3446).

2. Method according to claim 1, wherein the patients are sensitized to food, airborne or cutaneous allergens and, either, do not yet display allergic symptoms, or already display allergic symptoms.

3. Method of claim 1, wherein the non-replicating strain is administered orally or topically to the patient in a form selected from the group consisting of its pure form, in water, in milk, in breast milk, in a nutritional and in a topical composition.

4. Method according to claim 3, wherein the topical composition is in a form selected from the group consisting of a cream, lotion, gel, cream-gel, serum, ointment, and solution.

5. Method according to claim 3, wherein the nutritional composition is in a form selected from the group consisting of a food supplement, a human milk fortifier, or any milk support used during trophic feeding, an infant formula, a growing-up milk (powder or liquid), a nutritional dairy-based beverage (powder or liquid), a milk-based drink, a spoonable dairy-based food, a spoonable yoghurt-based food, a cereal-based milk drink, baby cereals, a yoghurt, a baby meal, a spoonable cheese-based food, dairy and fruit drink, a smoothy, a pudding, a snack, a biscuit and other bakery items.

6. Method according to claim 5, wherein the infant formula is in a form selected from the group consisting of a starter formula, a follow on formula, growing up milk, a partially or extensively hydrolyzed formula, or an amino acid based formula.

7. Method according to claim 1, wherein the non-replicating probiotic is administered in a nutritional composition wherein the composition comprises further ingredients or prebiotics.

8. Method according to claim 1, wherein the B. lactis NCC 2818 is administered in a daily dose of the equivalent of between 10⁵ and 10¹⁰ colony forming units (cfu) per day.

9. Method according to claim 1, wherein the composition is also comprising live B. lactis NCC 2818.

10. Method according to claim 1, wherein the symptoms are selected from the group consisting of gastro-intestinal, cutaneous, ocular, respiratory and combinations thereof.

11. Method according to claim 1, wherein the patients are young children under the age of six years old.

12. Method according to claim 1, wherein the patient is selected from the group consisting of children, teenagers and adults suffering from allergies.

13. Method according to claim 1, wherein the patients are non-human mammals suffering from allergies.

14. Method according to claim 1, wherein the symptoms are accompanied by modulation of one or more than one of tryptase, chymase, histamine, cytokines and leukotrienes in the patients.

15. (canceled)
